(19) United States
(12) Patent Application Publication
(10) Pub. No.: US 2007/0293933 A9
(43) Pub. Date: Dec. 20, 2007

(54) External support device

(76) Inventor: Mun Kyung Hong, New York, NY (US)

Correspondence Address: MARTIN D. MOYNIHAN d/b/a PRTSI, INC., P.O. BOX 16446, ARLINGTON, VA, 22215, US

(21) Appl. No.: 11/294,552

(22) Filed: Dec. 6, 2005

Related U.S. Application Data
(60) Provisional application No. 60/633,579, filed on Dec. 6, 2004.

(51) Int. Cl. A61F 2/06

(52) U.S. Cl. 623/1.12

(57) ABSTRACT

A medical device inserted into a blood vessel through a tube. The medical device comprises an external support device applied around a portion of the medical device in proximity to the tube.

### RELATED APPLICATION DATA

The present application claims the benefit under 35 USC 119(e) of U.S. Provisional Application No. 60/633,579 filed on Dec. 6, 2004 by Dr. Mun Hong and entitled “External Support Device”, the disclosure of which is incorporated herein by reference.

### FIELD OF THE INVENTION

The present invention relates to an external support device for a cardiac catheter.

### BACKGROUND OF THE INVENTION

Treatment of obstructed coronary blood vessels relies heavily on balloon angioplasty and/or stent delivery. These procedures typically employ a catheter which travels along a guidewire. In many cases navigation of the balloon or stent along a blood vessel to the target lesion is complicated by a tortuous path and/or calcified lesions. These obstacles can cause a physician to apply a degree of force to the catheter and/or guidewire which cause the catheter and/or guidewire to deform by kinking and/or bending. In many cases a deformed piece of equipment must be withdrawn and replaced.

Withdrawal and replacement significantly increase the cost of the procedure and require increased use of hospital resources and/or physician's time. Alternatively or additionally, withdrawal and replacement may expose the patient to additional risk.

Previously available alternatives include rotational atherectomy of heavily calcified vessels. This approach has not been completely successful and carries the risk of damage to the blood vessel from rotational atherectomy.

A number of US patents teach catheter anchors and/or holders which engage a catheter for the purpose of keeping it in position. Examples of this type of device may be found in U.S. Pat. Nos. 6,554,802; 5,368,575; 6,592,554 and 4,178,735. This list does not purport to be exhaustive. The disclosure of each of these patents is fully incorporated herein by reference.

A number of US patents teach catheter or guidewire straightening devices. Examples of this type of device may be found in U.S. Pat. Nos. 5,125,905; 5,282,479 and 4,840,613. This list does not purport to be exhaustive. The disclosure of each of these patents is fully incorporated herein by reference.

A number of US patents teach insertion devices and/or techniques. Examples of this type of device and/or technique may be found in U.S. Pat. Nos. 6,918,871; 6,309,374; 5,542,936; 6,830,561; 5,509,908; 4,767,409 and 4,569,347. This list does not purport to be exhaustive. The disclosure of each of these patents is fully incorporated herein by reference.

### SUMMARY OF THE INVENTION

An aspect of some embodiments of the present invention relates to an external support device (ESD) applicable to a catheter, guidewire or catheter/guidewire combination to prevent deformation thereof when opposing axial forces are applied during advancement towards a target lesion. Optionally, the ESD is supplied as a flexible sheet which is wrapped or folded around the catheter and/or guidewire. In an exemplary embodiment of the invention, the ESD is applied to a portion of the catheter and/or guidewire proximal to an aperture of a Y connector and the ESD advances towards and partially into the Y connector.

In an exemplary embodiment of the invention, an ESD is provided together with a catheter and/or Y connector as a kit.

In an exemplary embodiment of the invention, a method of advancing a medical device (e.g. catheter, guidewire or catheter/guidewire combination) into the body through a previously installed tube (e.g. insertion port and/or Y connector) includes supporting the medical device(s) with an ESD. Optionally, when a resistive force is encountered, the medical device is partially withdrawn to facilitate installation of the ESD. In an exemplary embodiment of the invention, the ESD shortens the length of the medical device in which deformation may occur. In an exemplary embodiment of the invention, the ESD is as long as the insertion port and/or Y connector. Optionally, this length permits the ESD to be inserted through the Y connector and/or port until it approaches a valve.

According to exemplary embodiments of the invention, there is provided a medical device inserted into a blood vessel through a tube, the medical device comprising an external support device (ESD) applied around a portion of the medical device in proximity to the tube.

Optionally, the ESD comprises a flexible sheet applied around said portion of the medical device by rolling.

Optionally, the ESD comprises ESD comprises at least two hinged elements adapted to engage said portion of the medical device.

Optionally, the flexible sheet comprises at least one support embedded in said sheet so as to parallel an axis of said medical device when said rolling occurs.

Optionally, the rolling of said flexible sheet around said portion of the medical device increases an outer diameter of said device in said portion to an increased outer diameter.

Optionally, the increased outer diameter is less than an inner diameter of said tube.

Optionally, applied around a portion of the medical device indicates less than one full circumferential turn around the medical device.

Optionally, applied around a portion of the medical device indicates at least one full circumferential turn around the medical device.

Optionally, said flexible sheet is characterized by a width of 10 mm to 30 mm and a length not exceeding 50 mm.

Optionally, the device is characterized by a thickness in the range of 1 mm to 8 mm.

According to exemplary embodiments of the invention, there is provided a kit comprising:

(a) a medical device adapted for insertion into a blood vessel through a tube; and

(b) an external support device (ESD) applicable around a portion of the medical device.

Optionally, the kit additionally comprises the tube.

Optionally, the ESD comprises a flexible sheet applicable around said portion of the medical device by rolling.

Optionally, the flexible sheet is characterized by a width of 10 mm to 30 mm and a length not exceeding 50 mm.

Optionally, the flexible sheet is characterized by a thickness in the range of 1 mm to 8 mm.

Optionally, the ESD comprises at least two hinged elements adapted to engage said portion of the medical device.

According to exemplary embodiments of the invention, there is provided a method of advancing a medical device into the body through a previously installed tube, the method comprising applying an external support device (ESD) to a portion of the medical device to which an axial force is to be applied.

Optionally, said applying includes rolling a sheet of flexible material about said portion of the device.

Optionally, the ESD increases a largest cross sectional dimension of said medical device by 1 mm to 3 mm.

Optionally, the method additionally comprises inserting an end of said ESD applied to said portion of the medical device into an aperture of said tube.

### BRIEF DESCRIPTION OF DRAWINGS

In the Figures, identical structures, elements or parts that appear in more than one Figure are generally labeled with the same numeral in all the Figures in which they appear. Dimensions of components and features shown in the Figures are chosen for convenience and clarity of presentation and are not necessarily shown to scale. The Figures are listed below.

FIG. 1 is a flow diagram illustrating a sequence of events associated with use of an external support device;

FIG. 2 illustrates a catheter/guidewire being inserted and becoming deformed when an obstruction is encountered (prior art);

FIGS. 3A and 3B illustrate an external support device (ESD) supplied as a flat sheet according to exemplary embodiments of the invention;

FIG. 3C illustrates an ESD according to FIG. 3A installed on a catheter/guidewire to prevent the deformation;

FIGS. 4A; 4B; 4C; 4D; 4E; and 4F are cross sections of an ESD according to exemplary embodiments of the invention applied to a catheter with 0.5 rotation, 0.8 rotation, 1 rotation, 2 rotations and 3 rotations about the catheter; and

FIG. 5 shows an ESD according to an exemplary embodiment of the invention supplied as a sterile object in a sealed package to preserve sterility.

### DETAILED DESCRIPTION OF EMBODIMENTS

Use of an External Support Device (ESD)

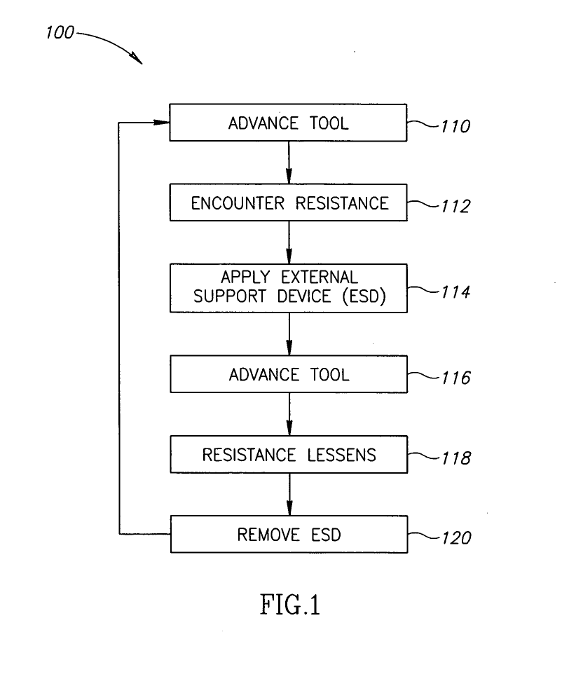

FIG. 1 illustrates a sequence of events associated with use 100 of an ESD. A medical device (e.g. catheter and/or guidewire) is advanced 110 through a blood vessel until resistance 112 is encountered. An ESD according to the invention is applied 114 to a portion of the device proximal to a Y connector. In an exemplary embodiment of the invention, the ESD permits application of a sufficient axial force to advance 116 the tool without deformation. Optionally, the ESD helps an operator direct the axial force in a desired direction. Optionally, application of the ESD reduces the size of a portion of the medical device in which deformation could occur. When resistance lessens 118 the ESD may be removed 120.

In an exemplary embodiment of the invention, the ESD is removed 120 by immobilizing the medical device at a point proximal to the ESD, optionally loosening the Y connector from the insertion port, and pulling the ESD away from the Y connector along the medical device. Leaving the ESD in place when not in use could possibly cause complications. Possibly, the complications include, but are not limited, to one or more of blood loss, inaccurate blood pressure measurement and air embolisms.

In an alternate exemplary embodiment of the invention, the ESD is removed 120 by advancing the ESD through the Y connector by pulling back the y-connector while holding the catheter in place.

In an exemplary embodiment of the invention support of a portion of a medical device by an ESD provides a method of advancing the medical device into the body through a previously installed tube such as a Y-connector. Optionally, the medical device with the ESD mounted upon it is insertable into the previously installed tube for a distance of 1 mm, optionally 5 mm, optionally 10 mm or lesser or intermediate or greater values. Optionally, an end of the ESD is inserted in a proximal end of the tube until it approaches a valve. Optionally, when a resistive force is encountered, the medical device is partially withdrawn to facilitate installation of the ESD. In an exemplary embodiment of the invention, the ESD shortens the length of the tool in which kinking may occur. Typically, kinking occurs between where the tool is restrained by the Y connector and/or blood vessel and a grasping point on the tool outside the Y connector. In an exemplary embodiment of the invention, the ESD is longer than the previously installed tube.

Physical Description of Exemplary Embodiments of an ESD

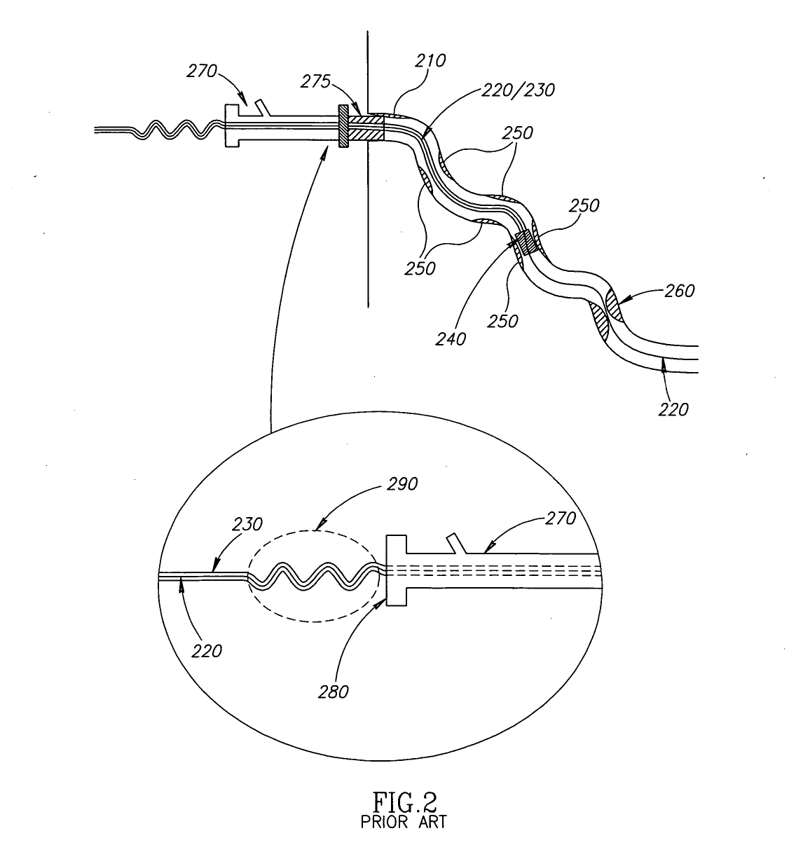

FIG. 2 illustrates a guidewire 220 and a catheter 230 inserted into a blood vessel 210 through a mouth 280 of a Y connector 270. Catheter 230 extends along guidewire 220 and has a medical tool 240 mounted at its distal end. In the illustration, medical tool 240 is a stent mounted on the outside of the catheter. Obstructions 250, for example calcified plaques and/or curves in blood vessel 210, cause resistance and make it difficult to navigate the medical tool and/or catheter 230 to target lesion 260. Guidewire 220, which is typically narrower than catheter 230 and/or tool 240 often reaches target lesion 260. According to previously available alternatives, application of additional axial force to overcome the resistance caused by obstructions 250 often resulted in deformation 290 of guidewire 220 and/or catheter 230, as shown in FIG. 2. Typically deformation 290 occurred in proximity to mouth 280 of Y connector 270.

According to various exemplary embodiments of the invention, methods and/or devices for prevention of deformation 290 are provided. Optionally, prevention is achieved by providing support to guidewire 220 and/or catheter 230 as axial force is applied.

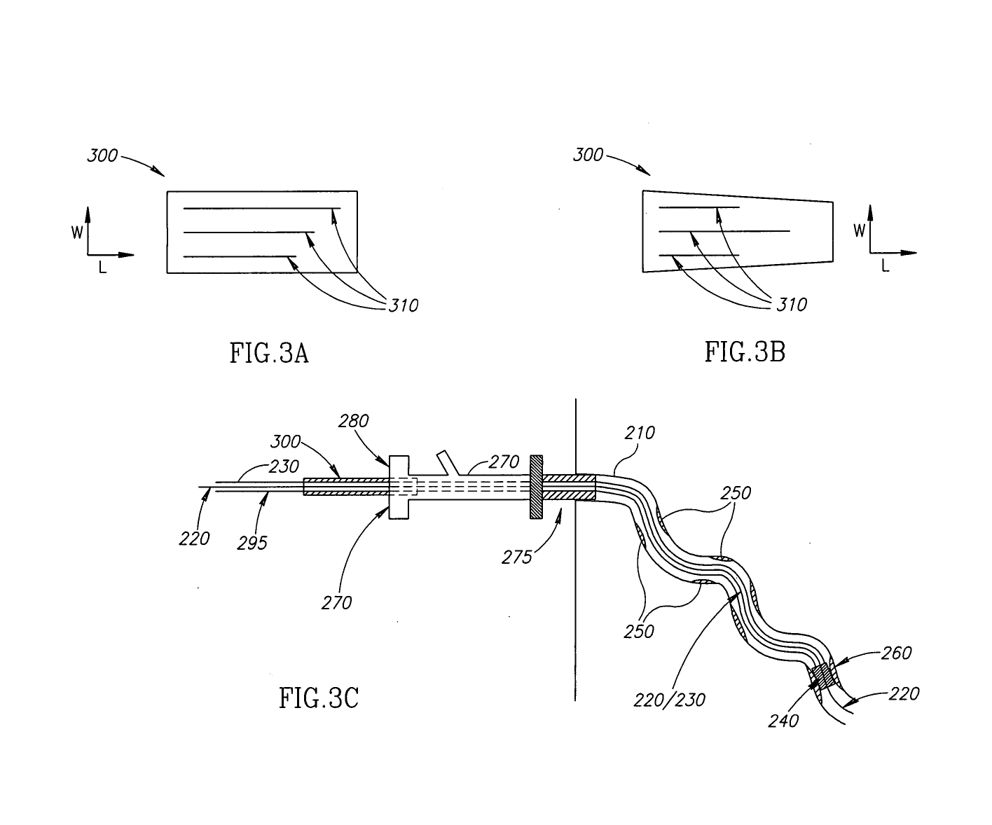

According to some exemplary embodiments of the invention (e.g. FIGS. 3A and 3B), an ESD is provided as a flat sheet which can be rolled around a portion of catheter 230 and/or guidewire 220.

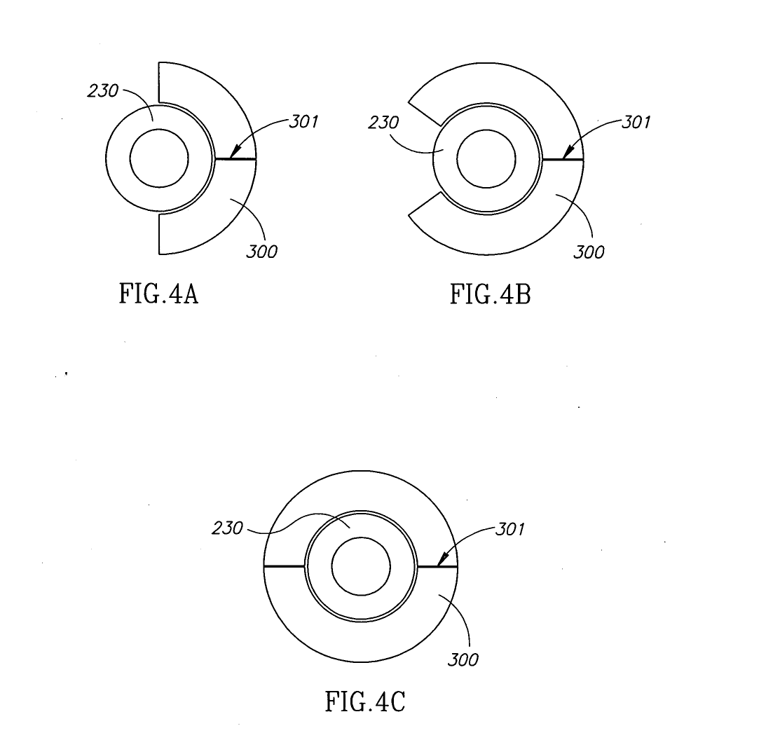

According to some exemplary embodiments of the invention (e.g. FIGS. 4A; 4B and 4C), ESD 300 includes a hinge 301 which permits the ESD to close and engage around a portion of catheter 230 and/or guidewire 220.

According to some exemplary embodiments of the invention, a flat sheet ESD 300 with a width (W) which is uniform along its length (L) is provided so that ESD 300 is characterized by a fixed W:L aspect ratio. According to these fixed W:L aspect ratio embodiments of the invention, ESD 300 forms a sleeve with a uniform circumference when it is wrapped around catheter 230 and/or guidewire 220. FIG. 3A shows an ESD 300 according to an exemplary fixed W:L aspect ratio embodiment of the invention. In an exemplary embodiment of the invention, ESD 300 is supplied as a flat sheet of flexible material. ESD 300 may be, for example rectangular, as pictured. Alternately, ESD 300 may be supplied as a square.

According to some exemplary embodiments of the invention, an ESD 300 with a width (W) which varies along its length (L) is provided so that ESD 300 is characterized by a variable W:L aspect ratio. According to these variable W:L aspect ratio embodiments of the invention, ESD 300 forms a sleeve with a non-uniform circumference when it is wrapped around catheter 230 and/or guidewire 220. In an exemplary embodiment of the invention, an end of the sleeve with a smaller circumference is introduced into a mouth 280 of Y connector 270. FIG. 3B shows an ESD 300 according to an exemplary variable W:L aspect ratio embodiment of the invention in which ESD 300 is supplied as a trapezoid. Additional examples of variable W:L aspect ratio embodiments of the invention include, but are not limited to, a semicircle and a triangle.

According to some exemplary embodiments of the invention, an ESD 300 with a width (W) which varies along its length (L) in two directions is provided so that ESD 300 is characterized by a variable W:L aspect ratio. Examples of ESD configurations characterized by a width (W) which varies along its length (L) in two directions include, but are not limited to, a circle, semicircle and an oval (not pictured). Rolling of a flat sheet with a width which varies along its length in two directions can produce a sleeve which is narrower at each end than in its central region. Optionally, this permits a user to apply ESD 300 to catheter 230 in one of at least two orientations.

Alternatively or additionally, a sleeve with a non-uniform circumference when it is wrapped around catheter 230 and/or guidewire 220 may be produced by applying an ESD with a fixed W:L aspect ratio at a desired angle so that a long edge of the ESD is not parallel to an axis of catheter 230 and/or guidewire 220.

Alternatively or additionally, a sleeve with a non-uniform circumference may be produced by employing a sheet of non-uniform thickness as described in greater detail hereinbelow.

Optionally, one or more rigid supports 310 (FIGS. 3A and 3B) are embedded in ESD 300. In an exemplary embodiment of the invention, supports 310 are deployed so that they will be parallel to a main axis of guidewire 220 and/or catheter 230 when ESD 300 is wrapped or rolled around guidewire 220 and/or catheter 230.

FIG. 3C shows ESD 300 of FIG. 3A rolled around a portion of guidewire 220 and/or catheter 230 in proximity to mouth 280 of Y connector 290.

FIGS. 4A; 4B; 4C; 4D; and 4E are cross sections of an ESD 300 applied to a catheter 230 with 0.5 rotation, 0.8 rotation, 1 rotation, 2 rotations and 3 rotations respectively about the catheter to form a rigid sleeve. According to some exemplary embodiments of the invention a sleeve formed by a single rotation (FIG. 4C), optionally as little as 0.8 rotations (FIG. 4B), optionally as little as about 0.5 rotations (FIG. 4A), provides sufficient support to catheter 230 to prevent deformation when an axial force is applied. According to alternate exemplary embodiments of the invention, a sleeve formed by two rotations (FIG. 4D) or three rotations (FIG. 4E) or more is employed to provide sufficient support to catheter 230 to prevent deformation when an axial force is applied.

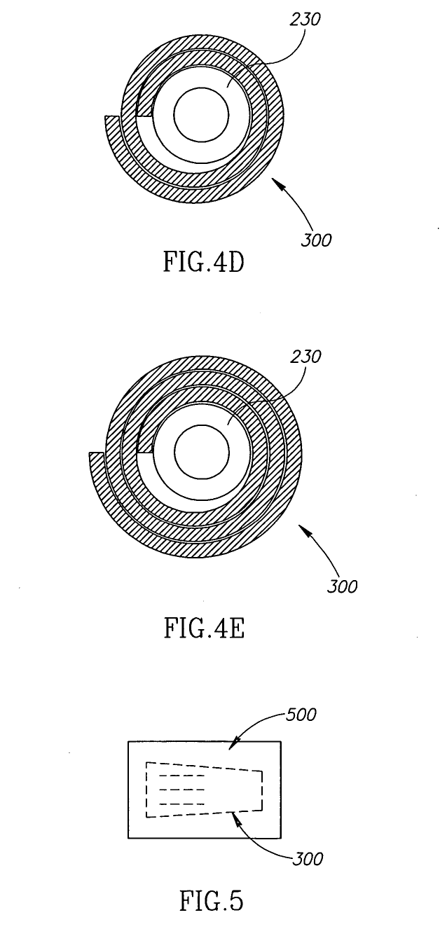

In an exemplary embodiment of the invention, an ESD 300 according to the invention is supplied in a sealed package (FIG. 5). Optionally, the sealed package 500 preserves sterility of ESD 300. ESD 300 and/or package 500 may be sterilized, for example, at a manufacturing facility or in a hospital. Sterilization may be, for example in an autoclave or by radiation means.

Referring again to FIG. 3B, optionally, ESD 300 is partially inserted into Y connector 290 during use. ESD serves as a gripping tool which distributes an applied axial force on guidewire 220 and/or catheter 230 along a length thereof. In an exemplary embodiment of the invention, any deforming force created by an applied axial force administered via ESD 300 is created inside ESD 300 and/or Y connector 290. Optionally, ESD 300 and/or Y connector 290 restrain catheter 230 and/or guidewire 220 from deforming. In an exemplary embodiment of the invention, insertion of ESD 300 into mouth 280 of Y connector 290 serves to focus the applied axial force in a desired direction. In an exemplary embodiment of the invention, any exposed portion of catheter 230 and/or guidewire 220 between ESD 300 and mouth 280 of Y connector 270 is sufficiently short to resist deformation.

In an exemplary embodiment of the invention, deformation is prevented because ESD 300 distributes applied force along guidewire 220 and/or catheter 230. According to previously available alternatives, a user of a catheter 230 would grip the catheter in two fingers and attempt to advance it towards a mouth 280 of Y connector 270. Typically, gripping occurred at some distance from mouth 280 of Y connector 270. Use of ESD 300 permits the user to more easily grip a length of catheter 230. Alternatively or additionally, use of ESD 230 permits application of axial force close to mouth 280 of Y connector 270, optionally within Y connector 270.

In an exemplary embodiment of the invention, deformation is prevented because the user is able to more accurately judge the direction of applied axial force by visual appraisal of the long axis of ESD 300 applied to catheter 230 and/or guidewire 220. Accurate judgment is optionally facilitated because ESD 300 is more prone to remain straight than the relatively flexible medical device. Accurate judgment permits correct direction of applied force along the axis of guidewire 220 and/or catheter 230. Theoretical explanations of the operative principle of devices and/or methods according to the present invention should not be construed as necessarily limiting the invention, but may be used for design decisions in some embodiments.

In the absence of deformation, a sufficient axial force may be applied to guide a tool (e.g. stent 240) past obstructions 250 to target lesion 260.

Once a decrease in resistance indicates that an obstruction 250 has been passed, ESD 300 may be removed. In an exemplary embodiment of the invention, in order to remove ESD 300, a portion of the medical tool (e.g. catheter 230 and/or guidewire 220) proximal to the ESD (e.g. 295) is immobilized. Immobilization may be achieved, for example, by pressing point 295 to an operating table or to a leg of the patient. Optionally, immobilization permits ESD 300 to be axially translated along the medical device away from blood vessel 210 and/or Y-connector 270. In an exemplary embodiment of the invention, Y-connector 270 is disengaged from port 275 and used to push ESD 300 away from blood vessel 210 along the medical device (e.g. catheter 230 and/or guidewire 220). Optionally, ESD may then be unrolled from the medical device. Optionally, Y-connector 270 may be re-engaged to port 275.

Construction Considerations

ESD 300 of some embodiments of the present invention may be constructed of any flexible material. In particular Hytrel® (DuPont; Wilmington, Del.; USA) may be employed to construct an ESD. Hytrel® is a thermoplastic polyester elastomer that provides a satisfactory degree of strength and stiffness while resisting tearing, creep, and abrasion. Hytrel® is amenable to common manufacturing processes, for example extrusion and/or roll pressing.

In an exemplary embodiment of the invention, a portion of the ESD which will contact the catheter and/or guidewire is softer and/or more flexible than a portion of the ESD which will form an outer portion of the sleeve when the ESD is rolled around the catheter and/or guidewire. It is a characteristic of Hytrel that temperatures used for extrusion and/or rolling influence the softness and/or flexibility of the Hytrel sheet being produced.

Support ribs 310, if employed, may be constructed of any rigid or semi rigid material such as, for example, nylon, metal or plastic. In an exemplary embodiment of the invention, support ribs 310 have a thickness which is less than an overall thickness of ESD 300 so that they may be embedded therein.

In an exemplary embodiment of the invention, ESD is supplied as a flexible sheet, optionally rectangular. Optionally, the sheet has a thickness of 0.25 mm to 8 mm, optionally 0.5 to 5 mm, optionally 0.75 to 3 mm, and optionally about 1 to 2 mm. Optionally, the thickness varies along a length of the ESD. In an exemplary embodiment of the invention, the thickness of the sheet varies by a factor of 2 along its length. According to this exemplary embodiment of the invention, the ESD is rolled around the catheter and/or guidewire with a thinner portion positioned closer to the Y connector and a thicker portion positioned further from the Y connector. Optionally, this contributes to formation of a sleeve which is narrower at an end closest to the Y connector and wider at an end furthest away from the Y connector.

Optionally, the sheet has a width of 10 mm to 30 mm, optionally 12 mm to 25 mm, optionally about 15 to 20 mm.

Optionally, the sheet has a length of 20 mm, optionally 30 mm, optionally 40 mm optionally 50 mm or lesser or greater or intermediate values. In an exemplary embodiment of the invention, the sheet has a length of 25 to 30 mm.

In an exemplary embodiment of the invention, the ESD is supplied as a sterile surgical tool. Optionally, the ESD may be supplied in a separate wrapping and/or as part of a catheter pack or kit contained in a sterile wrapper.

An ESD device according to the invention is a versatile tool. Versatility may stem, for example, from the ability of the ESD to conform to a wide variety of cardiac and/or angioplasty tools. A single ESD according to the invention may be suitable, for example, to use with one balloon shaft and one guidewire, with a monorail or over the wire balloon, or two wires and two balloon shafts as employed in a “bifurcation” procedure.

Regardless of the exact configuration of the tool to which the ESD is applied, the ESD with the angioplasty tool(s) securely inside it can be inserted into the Y-connector until a desired axial progress is achieved or there is a decrease in resistance. At this point the ESD may be removed. Optionally, the ESD may be applied to a different portion of the angioplasty tool(s) if necessary.

Optionally, the sleeve formed by wrapping the ESD around the angioplasty tool(s) increases a cross sectional aspect of the angioplasty tool(s) by 1 mm to 5 mm, optionally 1.5 mm to 3 mm, optionally 1.75 mm to 2.5 mm, optionally about 2.5 mm to 4 mm.

In an exemplary embodiment of the invention, an ESD applied to a catheter 230 and/or guidewire 220 increases a degree of rigidity of these medical tools by 20% or more, optionally 50% or more, optionally 100% or more, optionally 150% or more, optionally 200% or more, optionally 300% or more, optionally 500% or more. Optionally, an ESD 300 according to the invention which imparts a greater increase in rigidity permits a greater resistive force to be overcome.

Exemplary Modes of Deployment

In an exemplary embodiment of the invention, the ESD is rolled and or folded around the catheter. Optionally, the ESD surrounds the catheter 0.5 to 1 times. Optionally, the ESD surrounds the catheter 1 time, optionally 1.5 times, optionally 2 times, optionally 2.5 times, optionally 3 or more times. The exact number of times the ESD is rolled around the catheter may vary depending upon the materials employed and/or their thickness and/or a diameter of an aperture 280 of Y connector 270 the Y and/or a diameter of the catheter/guidewire.

In an exemplary embodiment of the invention, installation of an ESD on a catheter increases a largest cross sectional dimension of the catheter by 1 mm, optionally 1.5 mm, optionally 2 mm, optionally 2.5 mm, optionally 3 mm or more or lesser or intermediate or greater values.

In the description and claims of the present application, each of the verbs “comprise”, “include” and “have” as well as any conjugates thereof, are used to indicate that the object or objects of the verb are not necessarily a complete listing of members, components, elements or parts of the subject or subjects of the verb.

The present invention has been described using detailed descriptions of embodiments thereof that are provided by way of example and are not intended to necessarily limit the scope of the invention. In particular, numerical values may be higher or lower than ranges of numbers set forth above and still be within the scope of the invention. The described embodiments comprise different features, not all of which are required in all embodiments of the invention. Some embodiments of the invention utilize only some of the features or possible combinations of the features. Alternatively or additionally, portions of the invention described/depicted as a single unit may reside in two or more separate physical entities which act in concert to perform the described/depicted function. Alternatively or additionally, portions of the invention described/depicted as two or more separate physical entities may be integrated into a single physical entity to perform the described/depicted function. Variations of embodiments of the present invention that are described and embodiments of the present invention comprising different combinations of features noted in the described embodiments can be combined in all possible combinations including, but not limited to use of features described in the context of one embodiment in the context of any other embodiment. The scope of the invention is limited only by the following claims.

All publications and/or patents and/or product descriptions cited in this document are fully incorporated herein by reference to the same extent as if each had been individually incorporated herein by reference.

1. A medical device inserted into a blood vessel through a tube, the medical device comprising: an external support device (ESD) applied around a portion of the medical device in proximity to the tube.

2. A medical device according to claim 1, wherein said ESD comprises a flexible sheet applied around said portion of the medical device by rolling.

3. A medical device according to claim 1, wherein said ESD comprises at least two hinged elements adapted to engage said portion of the medical device.

4. A medical device according to claim 2, wherein said flexible sheet comprises at least one support embedded in said sheet so as to parallel an axis of said medical device when said rolling occurs.

5. A medical device according to claim 4, wherein said rolling of said flexible sheet around said portion of the medical device increases an outer diameter of said device in said portion to an increased outer diameter.

6. A medical device according to claim 5, wherein said increased outer diameter is less than an inner diameter of said tube.

7. A medical device according to claim 1, wherein applied around a portion of the medical device indicates less than one full circumferential turn around the medical device.

8. A medical device according to claim 1, wherein applied around a portion of the medical device indicates at least one full circumferential turn around the medical device.

9. A medical device according to claim 2, wherein said flexible sheet is characterized by a width of 10 mm to 30 mm and a length not exceeding 50 mm.

10. A medical device according to claim 2, characterized by a thickness in the range of 1 mm to 8 mm.

11. A kit comprising:
(a) a medical device adapted for insertion into a blood vessel through a tube; and
(b) an external support device (ESD) applicable around a portion of the medical device.

12. A kit according to claim 11, additionally comprising the tube.

13. A kit according to claim 11, wherein said ESD comprises a flexible sheet applicable around said portion of the medical device by rolling.

14. A kit according to claim 13, wherein said flexible sheet is characterized by a width of 10 mm to 30 mm and a length not exceeding 50 mm.

15. A kit according to claim 13, wherein said flexible sheet is characterized by a thickness in the range of 1 mm to 8 mm.

16. A kit according to claim 11, wherein said ESD comprises at least two hinged elements adapted to engage said portion of the medical device.

17. A method of advancing a medical device into the body through a previously installed tube, the method comprising applying an external support device (ESD) to a portion of the medical device to which an axial force is to be applied.

18. A method according to claim 17, wherein said applying includes rolling a sheet of flexible material about said portion of the device.

19. A method according to claim 17, wherein said ESD increases a largest cross sectional dimension of said medical device by 1 mm to 3 mm.

20. A method according to claim 17, additionally comprising inserting an end of said ESD applied to said portion of the medical device into an aperture of said tube.
